(19) United States
(12) Patent Application Publication
(10) Pub. No.: US 2011/0092956 A1
(43) Pub. Date: Apr. 21, 2011

(54) CATHETER WITH SEPARABLE SECTIONS

(75) Inventors: Wouter Anthon Soer, Eindhoven (NL); Maarten Marinus Johannes Wilhelmus Van Herpen, Eindhoven (NL)

(73) Assignee: KONINKLIJKE PHILIPS ELECTRONICS N.V., EINDHOVEN (NL)

(21) Appl. No.: 12/999,394

(22) PCT Filed: Jun. 24, 2009
(86) PCT No.: PCT/IB09/52710

§ 371 Date: Dec. 16, 2010

(30) Foreign Application Priority Data
Jun. 27, 2008 (EP) 08159250.3

(51) Int. Cl. A61M 25/00

(52) U.S. Cl. 604/528

(57) ABSTRACT

The present invention relates to a catheter comprising a catheter body section (1) and at least one catheter tip section (2). The catheter body section (1) can extend in a longitudinal direction. The at least one catheter tip section (2) may be located on a distal side of the catheter body section (1). In a first operation mode of the catheter usable during insertion thereof, the catheter body section (1) and the at least one catheter tip section (2) can be commonly moved substantially in the longitudinal direction. In a second operation mode of the catheter usable when the catheter has reached a position where it should not be advanced any further, the catheter body section (1) and the at least one catheter tip section (2) may be moved relative to each other, so that the at least one catheter tip section (2) can be separated from the catheter body section (1).

### FIELD OF THE INVENTION

The present invention generally relates to a catheter comprising a catheter body section and a catheter tip section separable from each other as well as a method of operating the same.

### BACKGROUND OF THE INVENTION

Minimally invasive surgery is commonly performed by inserting a catheter comprising e.g. surgical tools through a small incision into a cavity or duct of a patient's body. A major challenge in such operations is how to reach a target location if the path from an entry point or entry port at the incision to the target location comprises one or more sharp curves, or intersections allowing multiple possible paths (e.g. a Y-junction). Guidewires of a predefined shape are traditionally used to steer the catheter along a path where the amount of curvature and change of direction are limited. Furthermore, active catheters and active guidewires have been developed. They employ shape memory alloy actuators to bend a catheter along its path.

An inherent problem with these steering systems is that it is difficult to move a catheter along a path in which there is a large change of direction, e.g. of more than 90°, over a small radius of curvature. This is because the catheter can only be advanced towards the target location by pushing it through the entry port. Thus, with previously proposed catheter guidance methods it is difficult to move the catheter along a path in which there is a sharp curve.

U.S. Pat. No. 6,517,477 B1 discloses a catheter introducer system comprising a steering section and a propulsion section both located near a distal end of the catheter. The propulsion section is designed to pull the rest of the catheter through a body cavity or duct, so that there is no need to push the catheter along through the entry port. Propulsion is achieved by two or more gripping elements that can move relative to one another. The gripping elements attach to the wall of the cavity, e.g. by suction.

To achieve an optimal operation of the catheter introducer system proposed in U.S. Pat. No. 6,517,477 B1, the catheter should be flexible and rigid at the same time:

On the one hand, the catheter should be preferably very flexible in order for the propulsion system to be able to pull the catheter along its curved path; if the catheter is too stiff, very large forces (e.g. suction) are needed at the gripping elements, which may damage tissue to which they are attached.

On the other hand, the catheter should be preferably reasonably stiff so that it can be inserted close to the target location by pushing it through the entry port like a normal catheter; if the catheter is too flexible, the entire distance from the entry port to the target location must be covered by the propulsion mechanism, which is time-consuming and presents an unnecessary risk of tissue damage due to the many steps of gripping and releasing.

Due to the above contradicting requirements, the performance of the catheter introducer system disclosed in U.S. Pat. No. 6,517,477 B1 is not optimal.

### SUMMARY OF THE INVENTION

It is an object of the present invention to provide improved catheter guidance.

This object can be achieved by a catheter according to claim 1 and a method according to claim 14.

Accordingly, in a first aspect of the present invention, a catheter is presented. The catheter can comprise a catheter body section extending in a longitudinal direction and at least one catheter tip section located on a distal side of the catheter body section. The catheter body section and the at least one catheter tip section may be configured to be commonly moved substantially in the longitudinal direction in a first operation mode of the catheter. Further, the catheter body section and the at least one catheter tip section can be configured to be moved relative to each other in a second operation mode of the catheter, so that the at least one catheter tip section is separated from the catheter body section. Thus, the catheter may be inserted e.g. into a body cavity in a way similar to a normal catheter. Subsequently, the at least one catheter tip section can disengage from the catheter body section. Then, it may be steered and propelled independent from the catheter body section. This allows previously inaccessible locations to be reached and previously impossible actions to be performed. If a plurality of catheter tip sections configured to be moved independent from each other in the second operation mode is provided, even more complex functions than in case of using a single catheter tip section can be provided. For example, two catheter tip sections, both having a camera, may be detached from the catheter body section. This can allow to achieve a wider field of view or to generate a three-dimensional image. Different catheter tip sections may also have different functions. For example, one catheter tip section can have a camera, and another one may be provided with an electromagnetic sensor or a manipulating unit.

In a second aspect of the present invention, the at least one catheter tip section may be configured to be attached to a distal end of the catheter body section in the first operation mode and/or to be detached from the distal end of the catheter body section in the second operation mode. Thus, the at least one catheter tip section can be attached to the catheter body section when inserting or retracting the catheter, and may be detached from the catheter body section to be moved independent from the same when the catheter has reached its destination or a position where it cannot be advanced any further due to e.g. geometrical constraints.

In a third aspect of the present invention, the at least one catheter tip section can be configured to be steered and/or propelled independent from the catheter body section in the second operation mode. This allows previously inaccessible locations to be reached and previously impossible actions to be performed.

In a fourth aspect of the present invention, the catheter may further comprise at least one guiding unit connected to the at least one catheter tip section and configured to steer and/or propel the at least one catheter tip section. The at least one guiding unit enables to easily steer and/or propel the at least one catheter tip externally, e.g. from outside of a patient's body.

In a fifth aspect of the present invention, the catheter can further comprise a first sheath section connected to the at least one catheter tip section, extending in a hollow space of the catheter body section, and configured to slide out of the catheter body section in the second operation mode. Thus, the at least one catheter tip section may pull e.g. a flexible sheath out of the catheter body section. Hence, a working channel for an insertion of sensing units and/or manipulating units such as e.g. medical tools all the way through the catheter body section and the at least one catheter tip section can be provided.

In a sixth aspect of the present invention, the catheter may further comprise at least one gripping unit configured to grip and release a surface in an environment of the catheter, wherein the at least one gripping unit is arranged at the catheter body section and/or at the at least one catheter tip section. By means of the at least one gripping unit the catheter body section and/or the catheter tip section can be attached to the surface. This may enable steering and propulsion capabilities. Further, it can allow to provide a firm basis for examination or manipulation tasks, in which motion (e.g. due to heartbeat, respiration etc.) between a target and the at least one catheter tip section can be strongly reduced.

In a seventh aspect of the present invention, the at least one catheter tip section may comprise a respective first tip section and a respective second tip section, the first and second tip sections being configured to be moved relative to each other. The movement of the first and second tip sections relative to each other allows advancing the catheter tip section independent from the catheter body section so as to move it to previously inaccessible locations.

In an eighth aspect of the present invention, the catheter according to the seventh aspect can further comprise a second sheath section extending between the first and second tip sections. The second sheath section enables to keep a channel extending through the first and second tip sections to be kept closed when these sections are moved relative to each other.

In a ninth aspect of the present invention, the catheter according to the seventh aspect may further comprise at least one first guiding unit connected to the first tip section and configured to steer and/or propel the first tip section, and at least one second guiding unit connected to the second tip section and configured to steer and/or propel the second tip section. The at least one first and second guiding units allow to steer and propel the first and second tip sections independent from each other. This enables to advance one of these sections while the other one is kept at its position, in order to achieve a movement of the catheter tip section relative to the catheter body section.

In a tenth aspect of the present invention, the catheter according to the seventh aspect can further comprise at least one first gripping unit arranged at the first tip section and configured to grip and release a surface in an environment of the catheter, and at least one second gripping unit arranged at the second tip section and configured to grip and release a surface in the environment of the catheter. This allows to perform a sequence of gripping and releasing operations of the gripping units, wherein the first tip section may be advanced if the at least one first gripping unit is released and the at least one second gripping unit grips the surface, and the second tip section can be advanced if the at least one second gripping unit is released and the at least one first gripping unit grips the surface. Further, a firm basis for examination or manipulation tasks can be provided, in which motion (e.g. due to heartbeat, respiration etc.) between a target and the at least one catheter tip section may be strongly reduced. This can be achieved by attaching both of the first and second tip sections to the surface if the target has been reached.

In an eleventh aspect of the present invention, the catheter may further comprise at least one first channel extending through the catheter body section and the at least one catheter tip section and configured to guide at least one sensing unit and/or manipulating unit, and/or at least one second channel extending only through the catheter body section and configured to guide at least one sensing unit and/or manipulating unit. Thus, at least one working channel for an insertion of sensing units and/or manipulating units such as e.g. medical tools all the way through the catheter body section and the catheter tip section as well as at least one working channel for inserting such units only through the catheter body section may be provided. In this way, for example a surgical intervention can be performed using one of the working channels while a visual feedback at a desired viewing angle may be provided by a camera inserted through another one of the working channels.

In a twelfth aspect of the present invention, in the catheter according to the eleventh aspect the at least one first channel 6 can be at least partially enclosed by at least one sheath section 7, 8. Thus, a working channel for an insertion of sensing units and/or manipulating units such as e.g. medical tools through the catheter body section and the at least one catheter tip section may be kept closed when these sections are moved relative to each other.

In a thirteenth aspect of the present invention, the catheter can further comprise at least one sensing unit and/or at least one manipulating unit arranged at the catheter body section and/or at the at least one catheter tip section. As compared with an insertion of such units through channels extending through the catheter body section and the catheter tip section or only through the catheter body section, this can provide for even better stability with respect to the surface to which the catheter tip section is attached.

In a fourteenth aspect of the present invention, a method of operating a catheter comprising a catheter body section extending in a longitudinal direction and at least one catheter tip section located on a distal side of the catheter body section is presented. The method can comprise commonly moving the catheter body section and the at least one catheter tip section substantially in the longitudinal direction in a first operation mode of the catheter, and moving the catheter body section and the at least one catheter tip section relative to each other in a second operation mode of the catheter, so that the at least one catheter tip section is separated from the catheter body section. The method allows to insert the catheter e.g. into a body cavity in a way similar to a normal catheter. Subsequently, the at least one catheter tip section can be detached from the catheter body section. Then, it may be steered and propelled independent from the catheter body section. This enables previously inaccessible locations to be reached and previously impossible actions to be performed. Furthermore, motion (e.g. due to heartbeat, respiration etc.) between a target and the at least one catheter tip section can be strongly reduced.

In a fifteenth aspect of the present invention, a computer program is presented. The computer program may comprise program code means for causing a computer to carry out the steps of a method according to the fourteenth aspect when the computer program is carried out on a computer. Thus, the same advantages as with the method according to the fourteenth aspect can be achieved.

Further advantageous modifications are defined in the dependent claims.

### BRIEF DESCRIPTION OF THE DRAWINGS

These and other aspects of the present invention will be apparent from and elucidated by an embodiment described hereinafter, by way of example, with reference to the accompanying drawings, in which:

FIG. 1 shows a schematic diagram illustrating an exemplary catheter according to the embodiment in a first operation mode;

FIG. 2 shows a schematic diagram illustrating the exemplary catheter according to the embodiment in a second operation mode;

FIG. 3 shows a flowchart illustrating basic steps of an exemplary method according to the embodiment; and

FIG. 4 shows an example of a software-based implementation of the embodiment.

### DETAILED DESCRIPTION OF EMBODIMENTS

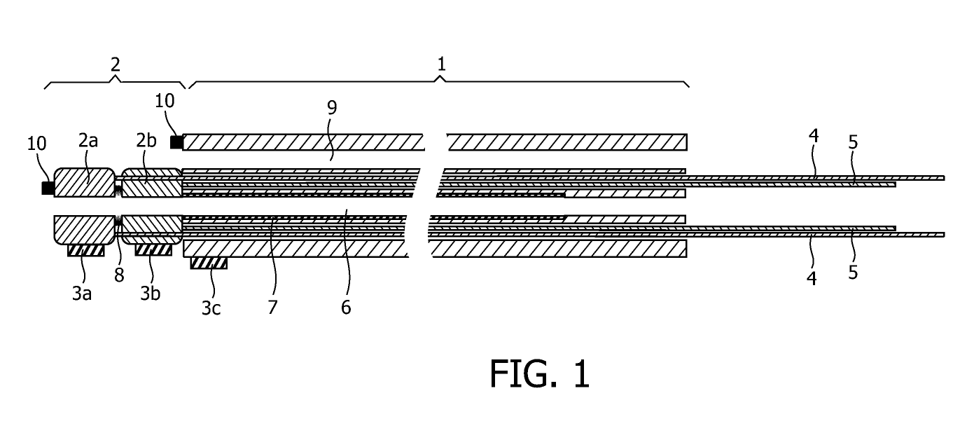

FIG. 1 shows a schematic diagram illustrating an exemplary catheter according to the embodiment in a first operation mode. FIG. 1 depicts a schematic cross section of the catheter. The catheter may comprise a catheter body section 1 and at least one catheter tip section 2 movable relative to each other. The catheter body section 1 can extend in a longitudinal direction thereof. The at least one catheter tip section 2 may be located on a distal side of the catheter body section 1 and can comprise a respective distal first tip section 2a and a respective proximal second tip section 2b movable relative to each other. A single catheter tip section 2 comprising one first tip section 2a and one second tip section 2b is illustrated in FIG. 1 and described in the following. However, a plurality of catheter tip sections 2 may be present.

Each of the first and second tip sections 2a, 2b can comprise at least one respective gripping unit or gripping element. That is, the first tip section 2a can include at least one first gripping unit 3a, and the second tip section 2b may include at least one second gripping unit 3b. Further, at least one additional gripping unit or element 3c can be provided at the catheter body section 1, e.g. at a distal end thereof. The gripping units 3a, 3b, 3c can be independently actuated to grip and release a surface in an environment where the catheter is operating. In case of a medical application, for example nearby tissue such as e.g. a wall of a cavity or vessel may be gripped and released. In case of another application, such as an inspection of a machine, some other surface like e.g. a wall of a machine part etc. can be gripped and released. The gripping units 3a, 3b, 3c may be actuated e.g. by suction, but can also be operated by other means such as e.g. synthetic gecko hair or inflatable cuffs. Several alternative ways of actuating gripping elements are described e.g. in WO 2005/046461 A1. Means for actuating the gripping units (e.g. flexible vacuum hoses in the case of suction elements) may pass through the catheter body section 1. However, they are not shown in FIG. 1 for the sake of clarity.

The gripping elements 3a, 3b, 3c enable steering and propulsion capabilities of the catheter tip section 2 and may be used to secure the catheter body section 1 and/or the catheter tip section 2 at a certain position as described in further detail below. While one gripping element 3a, one gripping element 3b and one gripping element 3c are depicted in FIG. 1, each of the first tip section 2a, the second tip section 2b and the catheter body section 1 may be provided with more than one gripping element.

The catheter can comprise at least one steering and propelling unit or guiding unit 4, 5 connected to the catheter tip section 2 or integrated with the same. The at least one guiding unit 4, 5 may be used to steer and/or propel the catheter tip section 2 and can be implemented in various ways. For example, a plurality of push-pull wires, cables and/or flexible drive shafts (described e.g. in WO 2005/046461 A1) extending through the catheter body section 1 may be employed. In order to provide steering capabilities in three dimensions, for example three guiding units 4 (two shown in FIG. 1) such as e.g. wires can be connected to the distal first tip section 2a or integrated with the same. They may pass freely through the proximal second tip section 2b and the catheter body section 1. Furthermore, for example three guiding units 5 (two shown in FIG. 1) such as e.g. wires can be connected to the proximal second tip section 2b or integrated with the same. They may pass freely through the catheter body section 1. In one example, the guiding units 4 and 5 can be wires made of a very elastic material such as nitinol. In another example, the guiding units 5 may be flexible sheaths enclosing wires 4.

The catheter can comprise a first channel 6 such as e.g. a tubular working channel for inserting sensing units and/or manipulating units such as e.g. medical tools to the front of the catheter tip section 2. The first channel 6 may be used to guide at least one sensing unit and/or manipulating unit. It can be enclosed respectively by the catheter body section 1, a first sheath section 7, the proximal second tip section 2b, a second sheath section 8 and the distal first tip section 2a, i.e. extend through respective hollow spaces of these sections. While one first channel 6 extending along a central axis of the catheter is depicted in FIG. 1, there may be more than one first channel 6, and the first channel 6 can extend adjacent to the central axis of the catheter.

The first sheath section 7 may be connected to the catheter tip section 2, i.e. to the proximal second tip section 2b, and can be e.g. a flexible sheath. If the distance between the catheter body section 1 and the catheter tip section 2 varies, the first sheath section 7 may slide into and out of the catheter body section 1 in order to keep the channel 6 closed over this distance. The catheter can comprise at least one stopper (not shown in FIG. 1) for the first sheath section 7. For example, stoppers may be provided e.g. at the distal end of the catheter body section 1 and the proximal end of the first sheath section 7 and can prevent that the first sheath section 7 slides out of the catheter body section 1 too far.

Similarly, the length of the second sheath section 8 may be variable in order to keep the channel 6 closed during a locomotion or movement of the catheter tip section 2. This can be achieved by using e.g. a folding sheath structure as shown in FIG. 1 or a sheath that can slide into one or both of the first and second tip sections 2a and 2b similar to the first sheath section 7. In case of employing e.g. a folding sheath structure, the second sheath section 8 may be connected to the first and second tip sections 2a, 2b. In case of employing a sliding sheath, stoppers can be provided e.g. at the first and second tip sections 2a, 2b as well as the second sheath section 8 in order to prevent that the second sheath section 8 slides out of the first and second tip sections 2a, 2b too far.

The catheter may comprise a second channel 9 such as e.g. a further tubular working channel. The second channel 9 can extend only through the catheter body section 1. It may be provided to insert sensing units and/or manipulating units such as e.g. medical tools through the catheter body section 1 but not through the catheter tip section 2. The second channel 9 can be used to guide at least one sensing unit and/or manipulating unit. While one second channel 9 extending through a peripheral portion of the catheter body section 1 is depicted in FIG. 1, there may be more than one second channel 9 located at e.g. various circumferential positions, and the second channel 9 can extend closer to the central axis of the catheter than depicted in FIG. 1.

In addition to the above applications of the first and second channels 6, 9, these channels may also be employed for guiding purposes. That is, e.g. guidewires can be used in the channels 6 and 9 (similar to normal catheters).

The catheter may comprise at least one sensing unit and/or manipulating unit 10 as described in the following. For example, the distal first tip section 2a of the catheter tip section 2 and/or the catheter body section 1 can comprise a camera or other means for visual feedback. If the catheter body section 1 comprises such image detection unit, it may be arranged e.g. at the distal end of the catheter body section 1. If the first tip section 2a is provided with a camera, for example a surgical intervention may be performed using working channels 9 of the catheter body section 1 while a visual feedback at a desired viewing angle can be provided by the camera at the first tip section 2a.

One or more electromagnetic sensors may be provided at the catheter tip section 2 and/or the catheter body section 1, e.g. at the distal end of the latter. Such electromagnetic sensors can be used for navigation during an intervention.

Further, an ultrasound probe may be provided at the catheter tip section 2 and/or the catheter body section 1, e.g. at the distal end of the latter. The ultrasound probe can be employed for intra-operative imaging.

Moreover, sensing units and/or manipulating units such as e.g. medical tools may be mounted at the distal first tip section 2a of the catheter tip section 2, rather than being fed through the first channel 6 or the second channel 9. This can provide for even better stability with respect to the surface to which the catheter tip section 2 is attached.

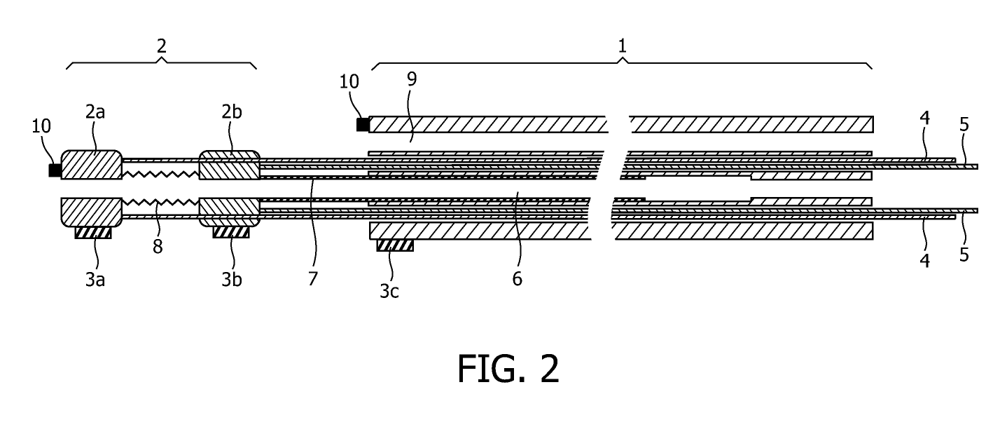

The above discussed sensing units and manipulating units may not only be provided at the first tip section 2a of the catheter tip section 2 and/or the catheter body section 1. In principle, they can also be provided at the second tip section 2b. Further, sensing units and manipulating units may not only be attached to the sections as shown in FIG. 1 and FIG. 2 but can also be integrated in the same.

In the first operation mode, the catheter tip section 2 may be attached to the catheter body section 1 as shown in FIG. 1. The first operation mode can be used e.g. to insert the catheter through an entry port. The entry port may be located e.g. at a small incision on a patient's body in case of applying the catheter for minimally invasive surgery, but it can also be located e.g. at an opening of a machine if performing a machine inspection or the like. In the following, a medical application of the catheter is described. However, the below description is to be considered illustrative or exemplary and not restrictive. Other application areas are possible, such as e.g. machine inspections and further utilizations where cavities and other areas not accessible from outside have to be examined, manipulated or otherwise treated.

If the catheter is inserted through the entry port at the patient's body, it can be advanced or moved forward into a body cavity by pushing it through the entry port. At this time, the catheter is in its first operation mode. In the first operation mode, the catheter tip section 2 may be attached to the catheter body section 1 as shown in FIG. 1. On the one hand, it can be kept in this position by a counter force exerted by the patient's body on the catheter tip section 2 during the insertion of the catheter. On the other hand, the catheter tip section 2 may be attached to the catheter body section 1 by keeping tension on the guiding units 5 so as to pull the proximal second tip section 2b against the catheter body section 1. In this stage, the catheter can be guided using the steering capabilities of the catheter tip section 2 to point the catheter in a desired direction. Alternatively, tension may be kept on the guiding units 4 to pull the distal first tip section 2a against the proximal second tip section 2b and both of them against the catheter body section 1, or tension can be kept on the guiding units 4 and the guiding units 5 at the same time.

When the catheter has reached its destination or a position where it cannot be advanced any further e.g. due to geometrical constraints, a second operation mode of the catheter can be used. FIG. 2 shows a schematic diagram illustrating the exemplary catheter according to the embodiment in the second operation mode. FIG. 2 depicts a schematic cross section of the catheter.

In the second operation mode of the catheter, the catheter tip section 2 may be detached from the catheter body section 1 and directed towards a target or target location along a soft tissue, for example a surface of an organ or an inner wall of a cavity or vessel. This can be effected by alternately advancing one section of the catheter tip section 2 while the other section is attached to the tissue. That is, a sequence of gripping and releasing operations of the gripping units 3a, 3b may be performed, the first tip section 2a can be advanced if the first gripping unit 3a is released and the second gripping unit 3b grips the tissue, and the second tip section 2b may be advanced if the second gripping unit 3b is released and the first gripping unit 3a grips the tissue. The first tip section 2a can be advanced e.g. by means of the first guiding units 4, and the second tip section 2b may be advanced e.g. by means of the second guiding units 5. A similar process is also described e.g. in WO 2005/046461 A1.

Thus, the first and second tip sections 2a, 2b can be alternatively advanced to move the catheter tip section 2 relative to the catheter body section 1 and towards the target. In this way, steering and propulsion capabilities of the catheter tip section 2 may be provided. The catheter body section 1 can be kept at its position in this stage or may also be moved. For example, it can be attached to nearby tissue while the catheter tip section 2 travels, in order to allow a more accurate guidance of the catheter tip section 2.

When the target has been reached, both of the first and second tip sections 2a, 2b may be attached to the tissue to provide a firm basis for endoscopy, surgical interventions or other operations. That is, both of the gripping elements 3a and 3b can be actuated to grip the tissue. Moreover, at least one additional gripping element 3c at the catheter body section 1 can also be actuated to further stabilize the position of the catheter.

If an endoscopy, a surgical intervention or another operation to be performed has been completed, the catheter may simply be removed in the following way. First, both of the first tip section 2a and the second tip section 2b of the catheter tip section 2 can be detached from the tissue. Then, the catheter tip section 2 may be retracted towards the catheter body section 1 by pulling the guiding units 5 and/or the guiding units 4. Finally, the entire catheter can be retracted from the cavity or other area. Thus, when the catheter is retracted, the catheter body section 1 and the catheter tip section 2 may be commonly moved. Hence, at least in this regard the catheter can be operated in the first operation mode when retracting it.

As described above, a catheter with a catheter tip section 2 that may be detached, steered and propelled externally is proposed. The catheter can be inserted e.g. into a body cavity in a way similar to a normal catheter. During the insertion, the steering capabilities of the catheter tip section 2 may be used to guide the catheter. Subsequently, the catheter tip section 2 can disengage from the catheter body section 1, attach itself to nearby tissue and travel further along this tissue. That is, the catheter tip section 2 may be detached and travel further to perform various types of functions. When detached, the catheter tip section 2 can pull a flexible sheath section, or a flexible catheter part, out of the (relatively stiff) main body of the catheter, i.e. the catheter body section 1. Thus, a working channel for an insertion of sensing units and/or manipulating units such as e.g. medical tools all the way through the catheter body section 1 and the catheter tip section 2 may be provided. This allows previously inaccessible locations to be reached and previously impossible actions to be performed. Furthermore, motion (e.g. due to heartbeat, respiration etc.) between the target and the catheter tip section 2 can be strongly reduced.

The above discussed catheter may also be regarded as a catheter consisting of a (normally) stiff proximal section (1) and a flexible distal section (2, 7). The flexible section can comprise the steering and propulsion system described above and may slide into and out of the stiff section. During catheter insertion, the flexible section is refracted, and the stiff section is used to get close to the target location. Subsequently, the flexible section extends from the stiff section as it is guided by the detachable catheter tip section 2.

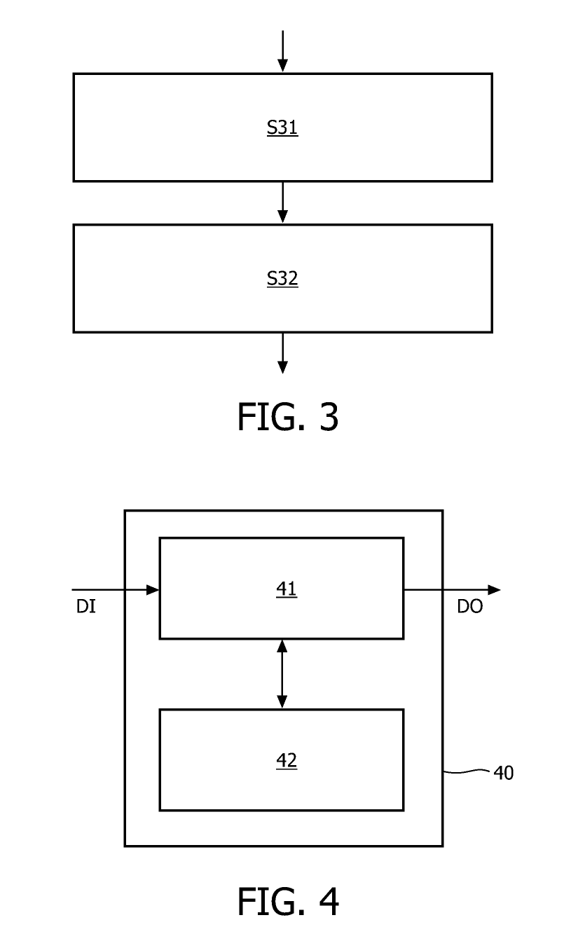

FIG. 3 shows a flowchart illustrating basic steps of an exemplary method according to the embodiment. The method comprises a step S31 of commonly moving a catheter body section 1 extending in a longitudinal direction and at least one catheter tip section 2 located on a distal side of the catheter body section substantially in the longitudinal direction in a first operation mode of a catheter, and a step S32 of moving the catheter body section and the at least one catheter tip section relative to each other in a second operation mode of the catheter, so that the at least one catheter tip section is separated from the catheter body section.

FIG. 4 shows an example of a software-based implementation of the embodiment. Here, a device 40 comprises a processing unit 41, which may be provided on a single chip or a chip module and which may be any processor or computer device with a control unit that performs control based on software routines of a control program stored in a memory 42. Program code instructions are fetched from the memory 42 and loaded into the control unit of the processing unit 41 in order to perform processing steps such as those described in connection with FIG. 3. The processing steps may be performed on the basis of input data DI and may generate output data DO.

In summary, the present invention relates to a catheter comprising a catheter body section 1 and at least one catheter tip section 2. The catheter body section 1 can extend in a longitudinal direction. The at least one catheter tip section 2 may be located on a distal side of the catheter body section 1. In a first operation mode of the catheter usable during insertion thereof, the catheter body section 1 and the at least one catheter tip section 2 can be commonly moved substantially in the longitudinal direction. In a second operation mode of the catheter usable when the catheter has reached a position where it should not be advanced any further, the catheter body section 1 and the at least one catheter tip section 2 may be moved relative to each other, so that the at least one catheter tip section 2 can be separated from the catheter body section 1.

While the present invention has been illustrated and described in detail in the drawings and foregoing description, such illustration and description are to be considered illustrative or exemplary and not restrictive. The invention is not limited to the disclosed embodiment. For example, while a single catheter tip section 2 is shown in FIG. 1 and FIG. 2 and described with reference to the same, a plurality of catheter tip sections 2 may be used. That is, a plurality of catheter tip sections 2 configured to be moved independent from each other in the second operation mode can be provided. Multiple catheter tip sections 2 of such plurality of catheter tip sections 2 may be employed for e.g. simultaneous observation and/or intervention operations.

For example, multiple detachable catheter tip sections 2 can be stacked behind each other and may be released one by one. When using multiple detachable catheter tip sections 2, more complex functions than in case of using a single detachable catheter tip section 2 can be achieved. For example, two catheter tip sections 2 may be detached from the catheter body section 1, both having a camera. This can enable to achieve a wider field of view or to generate a three-dimensional image. Different catheter tip sections 2 may also have different functions. For example, one catheter tip section 2 can have a camera, and another one may be provided with an electromagnetic sensor or a manipulating unit.

Further variations to the disclosed embodiment can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage.

A computer program capable of controlling a processor to perform the claimed features can be stored/distributed on a suitable medium, such as an optical storage medium or a solid-state medium supplied together with or as part of other hardware, but may also be distributed in other forms, such as via the Internet or other wired or wireless telecommunication systems. It can be used in conjunction with a new system, but may also be applied when updating or upgrading existing systems in order to enable them to perform the claimed features.

A computer program product for a computer can comprise software code portions for performing e.g. processing steps such as those described in connection with FIG. 3 when the computer program product is run on the computer. The computer program product may further comprise a computer-readable medium on which the software code portions are stored, such as e.g. an optical storage medium or a solid-state medium.

Any reference signs in the claims should not be construed as limiting the scope thereof.

1. Catheter comprising:
a catheter body section (1) extending in a longitudinal direction; and
at least one catheter tip section (2) located on a distal side of said catheter body section,
wherein said catheter body section and said at least one catheter tip section are configured to be commonly moved substantially in said longitudinal direction in a first operation mode of said catheter, and
wherein said catheter body section and said at least one catheter tip section are configured to be moved relative to each other in a second operation mode of said catheter, so that said at least one catheter tip section is separated from said catheter body section.

2. Catheter according to claim 1, wherein said at least one catheter tip section is configured to be attached to a distal end of said catheter body section in said first operation mode and/or to be detached from said distal end of said catheter body section in said second operation mode.

3. Catheter according to claim 1, wherein said at least one catheter tip section is configured to be steered and/or propelled independent from said catheter body section in said second operation mode.

4. Catheter according to claim 1, comprising:
at least one guiding unit (4, 5) connected to said at least one catheter tip section and configured to steer and/or propel said at least one catheter tip section.

5. Catheter according to claim 1, comprising:
a first sheath section (7) connected to said at least one catheter tip section, extending in a hollow space of said catheter body section, and configured to slide out of said catheter body section in said second operation mode.

6. Catheter according to claim 1, comprising:
at least one gripping unit (3a, 3b, 3c) configured to grip and release a surface in an environment of said catheter,
wherein said at least one gripping unit is arranged at said catheter body section and/or at said at least one catheter tip section.

7. Catheter according to claim 1, wherein said at least one catheter tip section comprises a respective first tip section (2a) and a respective second tip section (2b), said first and second tip sections being configured to be moved relative to each other.

8. Catheter according to claim 7, comprising:
a second sheath section (8) extending between said first and second tip sections.

9. Catheter according to claim 7, comprising:
at least one first guiding unit (4) connected to said first tip section and configured to steer and/or propel said first tip section; and
at least one second guiding unit (5) connected to said second tip section and configured to steer and/or propel said second tip section.

10. Catheter according to claim 7, comprising:
at least one first gripping unit (3a) arranged at said first tip section and configured to grip and release a surface in an environment of said catheter; and
at least one second gripping unit (3b) arranged at said second tip section and configured to grip and release a surface in said environment of said catheter.

11. Catheter according to claim 1, comprising:
at least one first channel (6) extending through said catheter body section and said at least one catheter tip section and configured to guide at least one sensing unit and/or manipulating unit; and/or
at least one second channel (9) extending only through said catheter body section and configured to guide at least one sensing unit and/or manipulating unit.

12. Catheter according to claim 11, wherein the at least one first channel (6) is at least partially enclosed by at least one sheath section (7, 8).

13. Catheter according to claim 1, comprising:
at least one sensing unit (10) and/or at least one manipulating unit (10) arranged at said catheter body section and/or at said at least one catheter tip section.

14. Method of operating a catheter comprising a catheter body section (1) extending in a longitudinal direction and at least one catheter tip section (2) located on a distal side of said catheter body section, said method comprising:
commonly moving said catheter body section and said at least one catheter tip section substantially in said longitudinal direction in a first operation mode of said catheter (S31); and
moving said catheter body section and said at least one catheter tip section relative to each other in a second operation mode of said catheter, so that said at least one catheter tip section is separated from said catheter body section (S32).

15. Computer program comprising program code means for causing a computer to carry out the steps of a method according to claim 14 when said computer program is carried out on a computer.
